(19) United States
(12) Patent Application Publication
(10) Pub. No.: US 2014/0216117 A1
(43) Pub. Date: Aug. 7, 2014

(54) Method for Reclaiming Usable Products from Biosolids

(71) Applicant: Nowa Technology, Inc., Prairie Village, KS (US)

(72) Inventors: Eugene F. DeShazo, Prairie Village, KS (US); Brian V. Yeh, Walnut Creek, CA (US)

(73) Assignee: Nowa Technology, Inc., Prairie Village, KS (US)

(21) Appl. No.: 14/094,391

(22) Filed: Dec. 2, 2013

Related U.S. Application Data
(63) Continuation-in-part of application No. 13/840,750, filed on Mar. 15, 2013.
Application No. 13/840,750 is a continuation-in-part of application No. 12/831,997, filed on Jul. 7, 2010, now abandoned.
(60) Provisional application No. 61/625,831, filed on Apr. 18, 2012.
(60) Provisional application No. 61/223,617, filed on Jul. 7, 2009.

(51) Int. Cl. C05B 17/00; C10L 1/02; C10M 175/00; C07C 67/58

(52) U.S. Cl. CPC C05B 17/00; C07C 67/58; C10L 1/02; C10M 175/0025
USPC 71/12; 554/177; 44/307; 508/111

(57) ABSTRACT

The method of reclaiming usable products from sludge is disclosed. A predetermined level of solvent within an extractor is heated, below boiling point, and dried sludge is immersed within the headed solvent. The solvent is a non-polar or polar aprotic solvents, such as ethyl acetate. The non-solid products, an oil/solvent mixture, within the sludge are separated and transferred to at least one evaporator with a concentration of between 5-25% oil in the solvent. The oil and solvent are is separated in one or more evaporators to remove approximately 80%-90%, and preferably 70%-95%, of the solvent. The solids are moved to a desolventizer for removal of the residual solvent and are then dried to a moisture content of below 25%, and preferably between 10 to 15%.

### CROSS REFERENCE

This application is a Continuation in Part of pending U.S. Ser. No. 13/840,750 filed Mar. 15, 2013 which is a non-provisional of U.S. 61/625,831 Filed Apr. 18, 2012 and pending U.S. Ser. No. 12/831,997 filed Jul. 7, 2010 which is a non-provisional of U.S. 61/223,617 filed Jul. 7, 2009, all 133 of which are incorporated herein as though cited in full.

### SUMMARY OF THE INVENTION

The disclosed invention relates to an improved method of reclaiming usable products, such as oil and fertilizer, from biosolids.

### BACKGROUND OF THE INVENTION

Wastewater sludge treatment and disposal cause some difficult and expensive challenges for municipalities with wastewater treatment systems. On average, about 6.5 million metric tons of sludge (on a dry basis) is produced each year in the U.S. alone (Water Environment Federation, 2008). This adds up to a disposal cost of more than $1 billion per year. As an example, the cities of Reno and Sparks, with a population of about 300,000 produce 30 million gallons per day of sewage, 120 tons per day of sludge and 18 tons per day of solids (dry basis).

A vast majority of that is either put in landfills, used as fertilizer, or incinerated, all of which are becoming increasingly expensive and cause various degrees of environmental concerns (Dufreche et. al., 2007). Another option, which has gained attention recently, is to use the processed sludge as an energy source. Different types of sludge have significantly different compositions. Primary sludge is taken from the initial filtering and settling and varies greatly in composition. Activated and secondary sludge are produced in aerobic digestion and contain bacteria and other microorganisms. Digested sludge is taken after an anaerobic digestion process. Since it contains anaerobic organisms which do not survive in climates with oxygen, digested sludge is a relatively benign substance which makes handling and storage easier. Several studies have examined extracting oils with a variety of solvents from different kinds of sludge for use in biodiesel production, all with limited effectiveness. This project explores the possibility of using digested sludge with alternative solvents as a source for extraction of oils, as opposed to types of sludge obtained from earlier in the sewage treatment process.

Various sewage treatment methods and plants are known in the art. Wastewater treatment operations use three or four distinct stages of treatment to remove harmful contaminants; according to the United Nations Environmental Program Division of Technology, Industry, and Economics Newsletter and Technical Publications Freshwater Management Series No. 1. “Biosolids Management: An Environmentally Sound Approach for Managing Sewage Treatment Plant Sludge” which goes on to say: “Each of these stages mimics and accelerates processes that occur in nature.

In the prior art a hexane/methanol/acetone solvent has been reported to extract 27.43 wt % of oils from activated sludge, but only 4.41 wt % of the activated sludge was saponifiable for production of biodiesel (Dufreche et. al., 2007). In-situ transesterification using methanol as an extraction solvent and reactant and sulfuric acid as a catalyst was reported to convert 14.5 wt % of biosolids in primary sludge into biodiesel (Mondala et. al., 2009). Another study reported yields of about 11.88 wt % of biodiesel from primary sludge using a Soxhlet extraction method and a hexane/methano/acetone mixture as the solvent (Willson et. al., 2010).

The removal of oil from waste, in this case hazardous wastes, was disclosed in U.S. Pat. No. 5,092,983 to Eppig et al. Eppig, however, requires the use of two solvents having defined dissolving ratios and boiling points, creating a complex and expensive system requiring additional equipment than a single solvent system. Eppig further teaches that the oil is extracted prior to contact with the first solvent

### BRIEF DESCRIPTION OF DRAWINGS

FIG. 1 is a flow chart depicting the entire process;

FIG. 2 is a flow chart depicting only the solvent in the process;

FIG. 3 is a flow chart depicting only the oil in the process;

FIG. 4 is a flow chart depicting only miscella to the first evaporation stage;

FIG. 5 is a flow chart depicting only the water in the process;

FIG. 6 is a graph of the extraction percentages using a Parr extractor;

FIG. 7 is a graph of the extraction percentages using a Soxhlet extractor;

FIG. 8 is a graph of the normal boiling curve;

FIG. 9 is a block flow diagram of mass balances during the extraction process; and

FIG. 10 is a diagram of the continuous countercurrent extractor;

FIG. 11 is a table of factorial design of experiment using 6 factors and 2 levels; and

FIG. 12 is an expanded table showing extraction result for FIG. 11.

### DETAILED DESCRIPTION OF THE INVENTION

### Definitions

As used in herein the term “biosolids” shall relate to the product generated from tertiary treatment of waste activated sludge as well as treated human waste

As used herein the term “miscella” shall relate to a solution of mixture containing an extracted oil or grease.

As used herein the term “DT” shall refer to a desolventizer-toaster

As used herein the term “DTD” shall refer to a unit containing a desolventizer-toaster and dryer cooler.

As used herein the term “POTW” shall refer to the publically owned treatment works as is used in the United States for a treatment plant that is owned, and usually operated, by a government agency. In the U.S., POTWs are typically owned by local government agencies, and are usually designed to treat domestic sewage and not industrial wastewater.

As used herein the term “about” means a range of +/−15%.

As used herein the term percent (%), means percent by weight.

Different types of sludge have significantly different compositions. Primary sludge is taken from the initial filtering and settling and varies greatly in composition. Activated and secondary sludge are produced in aerobic digestion and contain bacteria and other microorganisms. Digested sludge is taken after an anaerobic digestion process. Since it contains anaerobic organisms which do not survive in climates with oxygen, digested sludge is a relatively benign substance which makes handling and storage easier. For these reasons, it makes sense to use for oil extractions and was chosen as the focus of this research.

The term “biofuel feedstock” means organic material used in the production of biofuels.

The disclosed process provides numerous advantages over the prior art. First, it improves the quality of biosolids generated by wastewater treatment plants to enable its widespread use as a fertilizer product. The biosolids processed through the disclosed system are cleaner due to the solvent extraction removing oil, thereby containing minimal contaminants leaching out into the soil. This allows for wide spread use as a fertilizer and soil amendment. Further, removal of the oil makes the fertilizer hydrophilic.

Second, the solvent extraction and solvent removal step provides for multiple kill steps to eliminate, the pathogen level of the material, making it safer to handle. This is done without alkaline treatment, thus keeping the material at a neutral pH.

Third, the oily material that is removed can be used to provide heat to the process. As noted above, however, the quantity of extracted oil is dependent upon the contents of the sludge.

A fourth and essential feature is the efficient recovery of solvent that has a major positive impact on the economics of the process.

Finally the disclosed process is more economical to run than prior art designs and methods. The boiler can be the solitary heat source for the system, although outside heat sources could be used. The boiler can be run from the oil reclaimed from the sludge. Recovered solvent is fed to the solvent inlet of the extractor for reuse with a less than 500 parts per million solvent loss, giving a 99.6 solvent reuse. The expected steam consumption from this process is expected to be around 500 lbs. per ton of dry sludge processes. This takes into consideration the various heat exchange opportunities that are available based on a pinch analysis that was performed on the process. However, the amount of steam that is used in the process is also dictated and proportional to the amount of oil extracted from the incoming sludge. The 500 pounds of steam per ton reference is expected when the oil extracted is between 15-10% of the mass of the incoming sludge, however, if the oil contains around 7% oil, then the steam usage will drop to around 300 pounds of steam per ton. The reduction of steam is due to two factors: 1) the reduction of solvent required to extract the oil, and 2) the reduction of material that needs to be desolventized and distilled. Note that the relationship is not linear as there will be a minimum requirement for steam that is the threshold of the process.

The expected electrical consumption for the process is 25 kilowatt-hours per ton. Unlike steam consumption, the electrical consumption does not vary with oil concentration since it is energy that is used for conveying and is proportional to rate.

Currently the trend in the industry is for combined heat and power extraction for use in generating electricity as well as producing fertilizer. Due to the difficulties encountered, oil extraction is rare, or non-existent.

Wastewater sludge is the product of wastewater treatment plants and consists of matter consisting of municipal sewage and a more detailed list can be viewed on Wikipedia.org/wiki/Wastewater#Origin. And it is agreed that the breadth of the material fed into the process is broad, however it is submitted that it is consistent.

Because sludge is the aggregate of solids removed from wastewater in the treatment process, sludge is itself a waste product. Therefore, a certain variability exists. However, the range of that variability is relatively narrow. For example, all sludge contains about 25-36% metals and inerts (the remaining ash when a sludge sample is ashed at 500 deg. C.). All sludge contains between 5 and 12% extractable oil. (Note, on a laboratory basis, using a blend of solvents and a high temperature, high pressure extractor, more oil can be extracted, but the process is not scalable beyond the laboratory bench). See, Wastewater Engineering Treatment and Reuse, Metcalf and Eddy (Fourth Ed. 2003) pp. 1451-1457 (Pg. 1-7 Addendum A); Wastewater Sludge Processing, Turovskiiy and Mathai (2006), at p. 45 (Pg. 8-11 Addendum A); and Extraction of Lipids from Municipal Wastewater Plant Microorganisms for Production of Biodiesel, Dufreche, et. al., J Amer Oil Chem Soc (2007) 84:181-187¹.

The similarity of sludge is discussed in the book Wastewater Engineering Treatment and Reuse, Fourth Edition, by Metcalf & Eddy Inc., (Pg 1-7) wherein it is stated that untreated sludge and digested biosolids have a typical chemical composition, although quantities will differ to some extent. It should be noted that the publication by Metcalf & Eddy is considered the primary reference material for those involved in wastewater processes.

The sources for wastewater are predominately the same with some obvious variations based on local diet. Wastewater from New York City will have the same contents as Lewellan, Nebr. Quantities will differ however content will typically be the same.

Each facility can be customized to the contents of the sludge through testing at the time of design, to determine the time required for the process, including drying solvent immersion time, etc. A standard facility can be used in any location, however the process times can vary from facility to facility. The processed sludge from wastewater plants contains a reduced amount of bacteria. Any remaining bacterial is killed during the disclosed process, thereby producing a clean, environmental friendly fertilizer.

Equipment

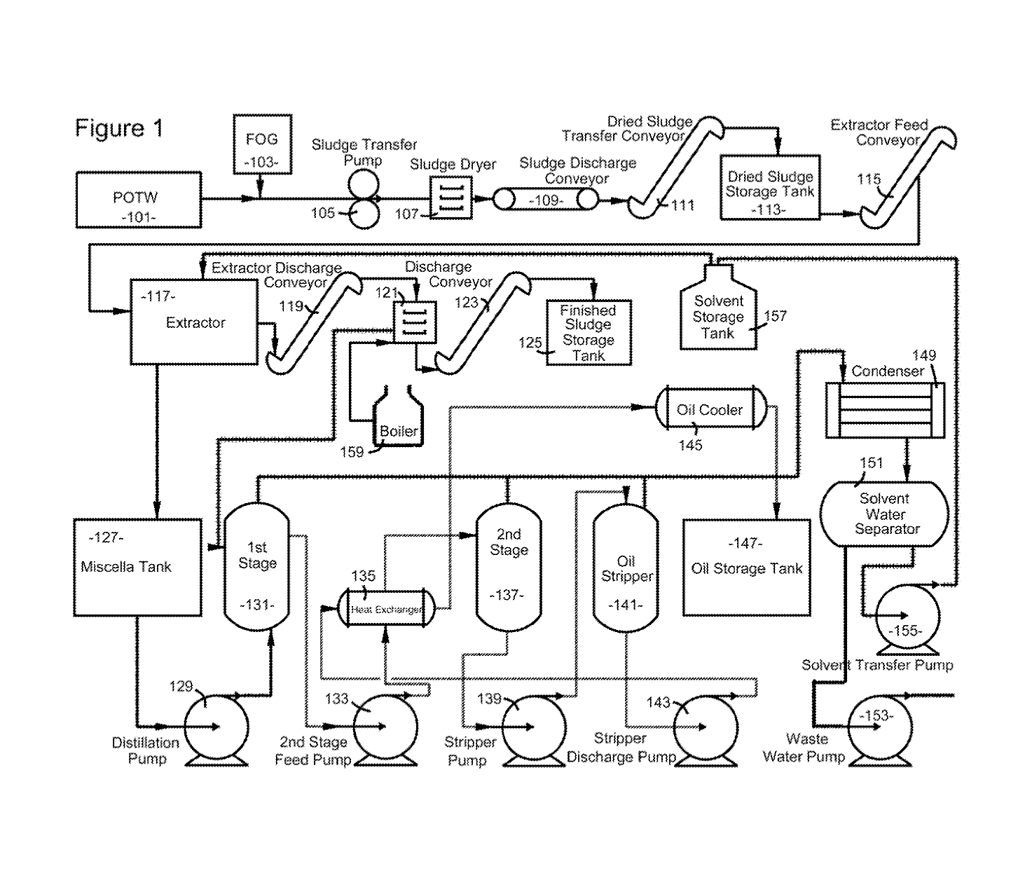

As shown in FIG. 1, sludge is generated at the POTW 101 by either anaerobic digestion or aerobic digestion of wastewater. At some plants, the generated sludge undergoes further anaerobic digestion to reduce the volume of sludge handled. Regardless of whether or not the sludge has undergone further digestion, the sludge can still be processed by the disclosed process. From the standpoint of the disclosed process, the processing point of the sludge does not matter so the sludge can be primary, secondary or tertiary.

Before the sludge leaves the POTW 101, it is dewatered to reduce the volume of the product. Belt presses are the most common dewatering devices in waste water treatment and can achieve anywhere between 10-35% solids (90%-65% moisture) after processing.

The second sludge source is the fat, oil, grease (FOG) 103. This includes animal fats, vegetable fats, and oils. A byproduct of cooking, FOG 103 comes from meat, fats, lard, oil, shortening, butter, margarine, food scraps, sauces, and dairy products. The FOG 103 is a solid or viscous substance, which will ultimately create an obstruction in the sewer system if not properly disposed. When washed down the drain, FOG sticks to the inside of sewer pipes. Over time FOG can build up, block entire pipes, and lead to serious problems.

The FOG 103 is often removed early in the processing of wastewater at the water treatment plant and treated separately through an anaerobic digester specifically designed to break down the FOG 103. However, using the disclosed process, this step may be skipped and the FOG 103 added directly to the sludge after it has been dewatered.

Sludge from the POTW 101 and FOG 103, if present, sources are fed to the sludge dryer 107 via the sludge transfer pump 105. At this point the sludge composition is in the range of 65% to 90% moisture.

The sludge proceeds to the sludge dryer 107 since to further process the sludge, it needs to be dried to anywhere between about 80-99% solids (20% to 1% moisture), preferably between 88-92% solids (8% to 12% moisture). There are many different dryer designs for drying sludge, such as a paddle dryer, ring dryer, flash dryer and equivalents as known in the field. For this process, a flash dryer, paddle dryer or hollow screw dryer are preferred due to energy efficiency and the ability to preserve the size of the particle. An example would be a flash dryer that will consume between 1300-1600 BTU per pound of water removed from the sludge. It should be noted, however, that the system will work with other driers that can be substituted that provide the equivalent results. In an alternative preferred embodiment, a fluidized bed dryer can be used because it is simple, maintains the integrity of the product, and is easier to operate. extraction is rate limited mass transfer with internal resistance to dissolution

The biosolid stays in the sludge dryer 107 for the length of time required to dry the biosolid to about 10-15% moisture. A moisture content between 25-30% moisture does not produce consistent results with respect to oil recovery. The length of time will be dependent upon the content of the sludge, as well as size and type of dryer. Drying to a moisture content of below 10% has been found to be economically inefficient.

The dried sludge is preferably a granular product having granules as described in detail hereinafter.

The dried biosolid is transferred away from the sludge dryer 107 via the sludge discharge conveyor 109. There are many ways of conveying and depending on the layout, multiple conveyors can be used. For example, in the present embodiment the dried biosolid is transferred to a second conveyor, a dried sludge transfer conveyer 111. The conveyor moves the sludge onward to the dried sludge storage tank 113. Alternatively the dried sludge could be transported directly from the dryer 107 to the storage tank 113. The method of transporting the sludge will be known to those in the art.

The dried sludge storage tank 113 stores the sludge rather than moving it straight to the next process to act as a buffer between processes. As some parts of the system are processed through faster than others, the dried sludge storage tank 113 prevents the later processes from being overloaded with too much sludge. This enables further steps to take from the bin on an “as needed” basis. The dried sludge can stored anywhere from 15 minutes to indefinitely, depending on delivery, remaining equipment and work schedules, before moving to the next process. The dried sludge storage tank 113 size is in the range necessary to detain between 15 minutes to three days of dried sludge production. The dried sludge storage tank 113 is preferably vented to release moisture, is preferably lined or clad with a corrosion resistant material, and it preferably has an unloading device on the bottom to assist in removing the material should the material be susceptible to bridging.

The dried sludge is transferred away from the dried sludge storage tank 113 via the extractor feed conveyor 115 or other applicable transportation means. The conveyor moves the sludge to a continuous solvent extractor 117.

The solvent extractor 117 removes the oil and any impurities that could have leached into the soil and aids in the destruction of pathogens. The extraction time is between 0.5-6 hours with 4 hours being preferred. The extraction time is determined by the size of the dryer and the contents of the sludge. A continuous counter-current immersion solvent extractor is the preferred embodiment, however there are other possible alternative embodiments. The extractor can be batch or continuous, although continuous is preferred as it reduces equipment costs and operates at a lower temperature which reduces energy costs as well as startup costs. The extractor may be a co-current or countercurrent, although countercurrent is the preferred embodiment. The countercurrent extraction uses multiple stages of liquid-solid separations to separate the oil from the solvent. Through this multistage process, even if the separation at each stage is small, the overall system can have a high separation output. The countercurrent extraction is such that the flow of the solvent travels in the opposite direction than the flow of the sludge is traveling.

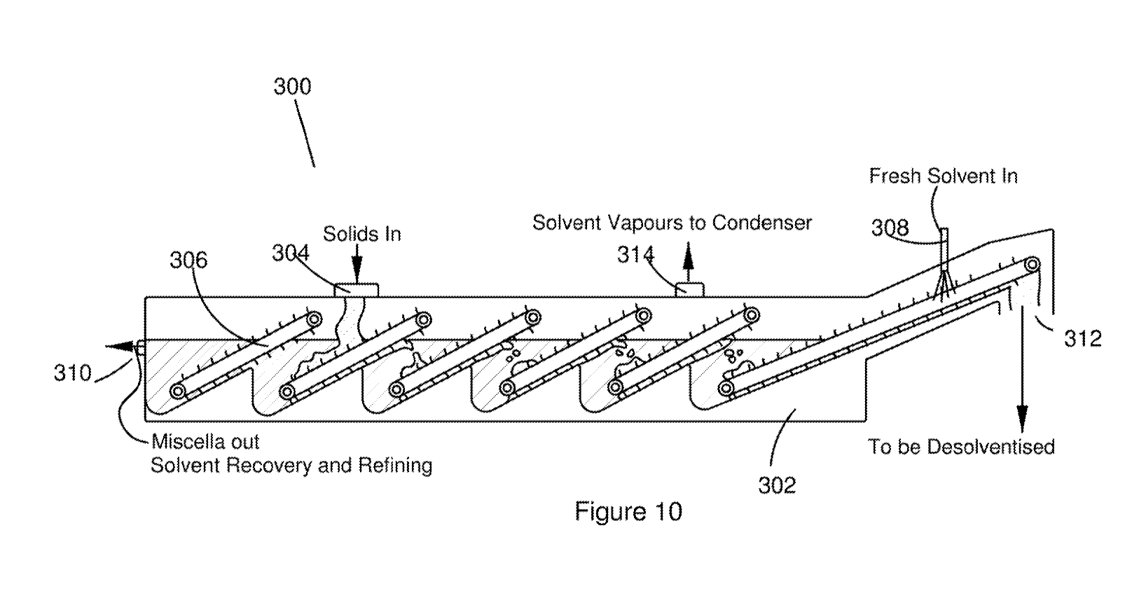

An example of a continuous countercurrent extractor 300 is illustrated in FIG. 10. The solvent is added up to level that will maintain the sludge submerged during the process, prior to the sludge being moved into the extractor. The solids enter the extractor chamber 302 where contact is made with the belts 306 which, in this illustration would be rotating clockwise. The sludge moves along the belts 306, dropping with each sequential belt. The number of belts will be dependent upon the size of the operation and although 6 are illustrated in this Figure, more or fewer belts can be used. The solvent input port 308 is raised from the chamber 302 and configured so that the current of the solvent entering the chamber 302 is going in the opposite direction (counter current flow) from the sludge. The majority of the solvent is recovered for reuse in the system, thereby reducing the costs of operation.

The vapors from the solvent exit the vapor port 314 for the condenser and subsequent recovery. The miscella leaves through the miscella port 310 for subsequent solvent recovery. The solvent containing solids is discharged the desolventizer port 312.

Alternatively, the extractor can be a percolation type, although immersion is the preferred embodiment. An example of an immersion device is the Model IV manufactured by the Crown Iron Works at Minneapolis, Minn. and Model V being an example of percolation extraction. Additional data on reclaiming oil and fertilizer using a percolation extractor system can be found in co-pending application Ser. No. 12/831,997, filed Jul. 7, 2010 which is incorporated herein by reference. An immersion extractor is easier to operate than other forms of extractors, and can handle all levels of sludge as well as all particle sizes.

The solvent can be any organic solvent, such as hexane, heptane, acetone, ethyl acetate, methyl-iso-butyl ketone, butanol, chloroform or others well known to industry. It has been found, however, that ethyl acetate produces the greatest percentage of oil with minimum power expenditure. Water, as well as benzene and other extreme polar solvents, will not extract the oil from the sludge.

The extractor is designed to be able to be set to an operation temperature at less than the atmospheric boiling point of the solvent of choice. Although close to, or 10-20 degrees below the boiling point of the solvent produces the highest oil output, the gain rapidly diminishes with increasing temperature. For example it has been found that hexane, which boils at 156 degrees F., extracts more oil at 140 than at 70 degrees F.

The rate of solvent addition is such that a concentration between 5%-25% oil in the solvent is achieved. The preferred concentration is between 13%-20%. The extractor can have a mechanism to allow for gravity flow dewatering to occur for any additional moisture before the solids are discharged into the extractor discharge conveyor 119. The solvent content of the solids upon exiting the extractor is between 10-30% solvent.

The liquid that exits the extractor is known as miscella and contains between 13-20% solvent soluble materials (oil). The liquid flows into a tank known as the miscella tank 127 where it is held prior to distillation. The distillation process takes is generally under 5 hours with 30-60 minutes being preferred. The tank size commonly used for oil production would most likely complete distillation in the range of 2-4 hours, although smaller or larger tanks can be used. The size of the tank would depend upon the size of the plant, work schedules, etc. The materials separated by the distillation are the oil contained in the sludge and the solvent. After distillation the oil is free of solvent.

Once the extraction process is completed, the sludge goes on to the extractor discharge conveyor 123 towards the DT 121. As stated heretofore, the oil remaining after extraction can optionally be further refined in the extractor processes until it is sent to the miscella tank 127.

The distillation pump 129 serves the purpose of transferring the oil/solvent mixture into the 1^{st} stage evaporator 131.

After the miscella tank 127, the miscella is pumped, through use of a distillation pump 129, into the 1^{st} stage evaporator 131, such as a still, rising film evaporator, and equivalent equipment that can remove the solvent from the oil. The 1st stage evaporator 131 serves the purpose of utilizing waste heat from the desolventizer and/or boiler heat to separate about 70%-95%, with optimally 80%-90% of the solvent from the oil/solvent mixture. Any type of evaporator can be used including still, rising film, falling film, wiped film and short path. In a rising film evaporator, boiling takes place inside the tubes, due to heating (usually by steam) of the outside of the tubes. With this process submergence extraction is therefore not required; as the creation of water vapor bubbles inside the tube creates an ascensional flow enhancing the heat transfer coefficient. This type of evaporator is therefore quite efficient, the disadvantage being to be prone to quick scaling of the internal surface of the tubes. Tubes are usually quite long (4+ meters) and sometimes a small recycle is provided. Sizing this type of evaporator is usually a delicate task, since it requires a precise evaluation of the actual level of the process liquor inside the tubes. Further details regarding evaporation are found in U.S. Pat. No. 5,582,692 which is incorporated by reference herein.

Heat to the 1^{st} stage evaporator 131 is provided by the hot vapors coming out of the boiler 159, which can or cannot pass through desolventizer 121 depending upon plant design. Once the latent heat from the hot vapors is recovered, the condensed vapors flow from the 1^{st} stage evaporator 131 to the solvent water separator 151, where the solvent and water are separated. The solvent is then transferred, via the solvent transfer pump 155, back to the storage tank 157 for reuse. The water is sent to waste water disposal pump 153 and then back to the boiler 159 for reuse or alternatively to the POTW 101 or other disposal areas. In the head section of the 1^{st} stage evaporator 131, the solvent vapors travel to a condenser 149 where it is condensed prior to being sent to the solvent water separator 151. The remaining miscella leaves the evaporator containing on average about 75-85% oil and 25-15%, and generally 80% oil and 20% solvent. The oil/solvent percentages will vary based upon the type of evaporator.

The 2nd stage feed pump 133 serves the purpose of transferring the oil/solvent mixture from the 1^{st} stage evaporator to 2^{nd} Stage evaporator. Prior to entering the 2^{nd} stage evaporator, the mixture goes through a heat exchanger 135. The heat exchanger 135 preheats the feed into the 2^{nd} stage evaporator 137 with the oil from the stripper 141 to ensure that the mixture remains in vaporous stage. This heat recovery increases the temperature of the miscella to the degree required to maintain this vaporous stage, which is generally by about 50 F The oil cooler 145 cools the oil from the heat exchanger 135 with cooling water, taking the oil from a temperature of approximately 140-200 F to a temperature of approximately 100˜130 F. The cooling water is brought into the equipment from any available, applicable source.

After cooling the oil has completed its processing and is stored in the oil storage tank 147. The oil can then be used to as heat for the process, a bunker fuel, asphalt enhancer, lubricant, to supplement crude oil or for any other use depending on the purity of the oil recovered. The oil composition is dependent on the quality of sludge and can vary greatly.

The 2nd stage evaporator 137 serves the purpose of further separating the solvent from the oil. As with the 1st stage evaporator 131, any type of evaporator may be used including rising film, still, rising film, falling film, wiped film and short path. Heat to the evaporator 137 is provided by plant steam or outside sources. As with the solvent vapors from the 1^{st} stage evaporator 131, the vapors from the 2^{nd} stage evaporator travel to the condenser 149 where it is condensed, and then transferred to the solvent water separator 151. The remaining miscella leaves the evaporator containing about 97%-99% oil and 1%-3% solvent.

The miscella from the 2nd stage evaporator 137 then travels to the oil stripper 141; powered by the stripper feed pump 139. In the oil stripper 141, the miscella travels counter current to sparge steam that is used to strip away the remaining solvent with the solvent riding up on the steam out of the oil stripper 141. Different internal designs for the oil stripper 141 may be used including random packing, sieve tray and disk and donut. In this system, a disk and donut configuration is preferred. The oil is discharged out of the oil stripper 141 containing less than about 500 parts per million of solvent. Solvent vapors from the oil stripper 141 travel to the condenser 149 where it is condensed, and then goes to the solvent water separator 151. Due to the very low remaining solvent, roughly 99% of the solvent used in the process is recovered. Further details regarding oil stripping using disc and donut is found in U.S. Pat. Nos. 3,503,854 and 6,703,227, which are incorporated by reference herein.

The stripper discharge pump 143 serves the purpose of removing the oil from the stripper 141. The materials are processed back to the heat exchanger 135 and then onto the oil cooler 145 and storage tank 147 The resulting oil may be used directly in a boiler 159 for generating heat within the system of commercial uses as described heretofore.

The dried sludge is transferred away from the extractor 117 via the extractor discharge conveyor 119. The conveyor moves the sludge to the desolventizer 121. At this point the dried sludge is of the composition of about 30% solvent and about 70% percent oil free sludge.

The desolventizer 121 which serves the purpose of removing the solvent from the sludge and drying and cooling the sludge so that it is suitable for storage Although a single desolventizer unit is illustrated herein, separate units, with transferring means between the DT and DC, can be used. The solids are desolventized using an apparatus commonly known in the oilseed industry as a desolventizer-toaster, or equivalent. The apparatus uses a combination of agitation, indirect heat and a condensable inert gas as a stripping medium. In this system, steam, which comes from a boiler 159, is the preferred stripping gas. The operating temperature of the desolventizer-toaster 121 is preferably between about 220-250 F and with the sludge remaining in the desolventizer a mean residence time between about 15-30 minutes, or until the desired moisture content is reached. The solids leaving the desolventizer, or alternatively DT, preferably contain no more than 300 ppm of solvent, and will have a moisture content between about 5-20%. Further details regarding DTDC and general desolventizers are in U.S. Pat. No. 5,992,050 which is incorporated by reference herein.

In some embodiments, such as the example illustrated herein, after the desolventizer 121, the solids will flow into a DC. The DC allows for heated air to further dry the material and is followed by a flow of ambient air to cool the material before storage.

The dried sludge is transferred away from the desolventizer 121 via the discharge conveyor 123. The conveyor moves the sludge to the finished sludge storage tank 125.

At the finished sludge storage tank 125 the sludge is of the composition of about 90% sludge and 10% moisture. The biosolids are cleaner and pathogens eliminated, meaning there will be no pathogens leaching out into the soil and is thus is safe to handle. Meets Class A Exceptional Quality Biosolides as set by Code 40 CFR part 503. The final sludge is used for a high value fertilizer/soil amendment.

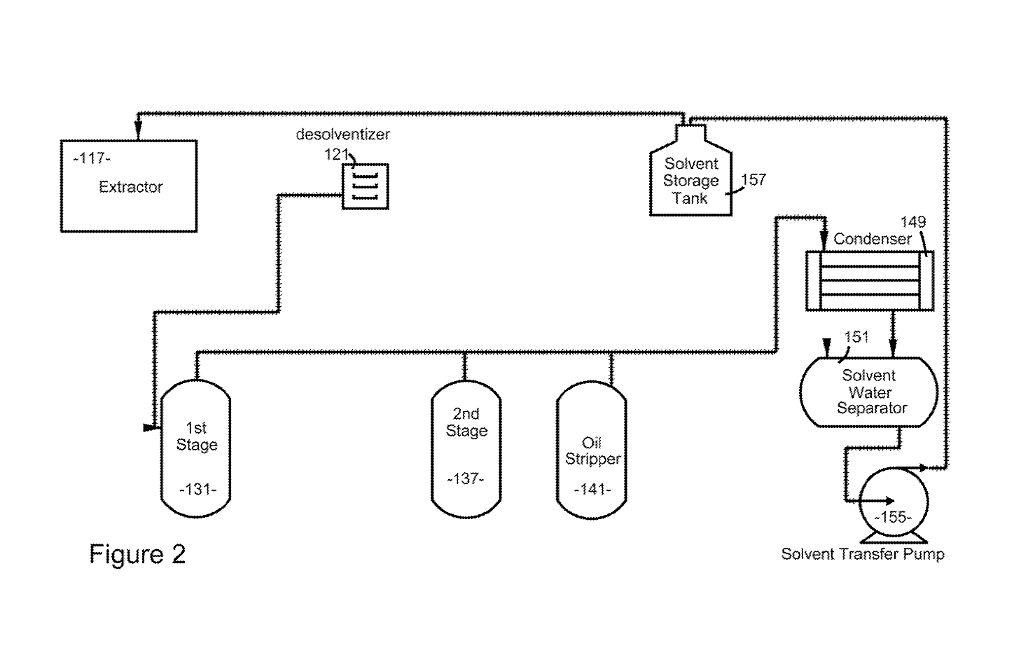

FIG. 2 shows in more detail only the solvent portion of the system. The solvent starts in the solvent storage tank 157 and enters the process at the extractor 117. The solvent also enters the 1st Stage evaporator 131 from the desolventizer 121 and the extractor 117. The solvent then proceeds from the 1st Stage evaporator 131 to the 2nd Stage evaporator 137, the Oil Stripper 141, the condenser 149 and the solvent water separator 151. From the solvent water separator 151 the solvent goes to the solvent transfer pump 155 back to the solvent storage tank 157.

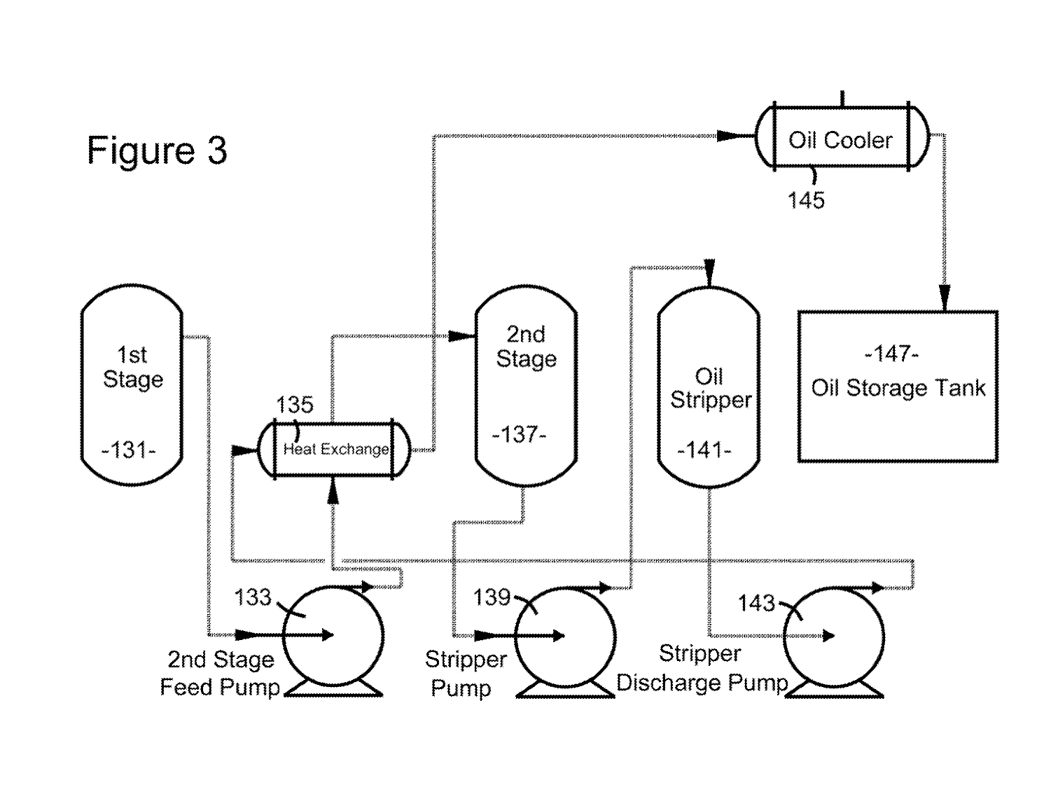

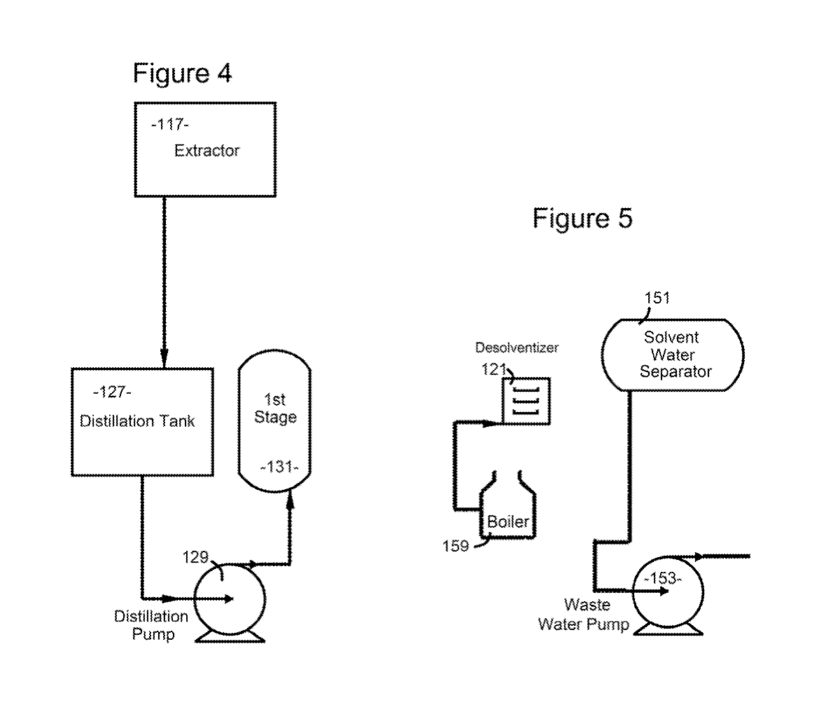

FIG. 3 shows the path of the oil starting at the 1st Stage evaporator 131 going through the 2nd stage feed pump 133 to the heat exchanger 135. From there it either goes from the 2nd stage evaporator 137 on to the stripper pump 139 then to the oil stripper 141 to the stripper discharge pump 143 and back to heat exchanger 135 to the oil cooler 145 to the final oil storage tank 147. FIG. 4 shows the first evaporation stage of the miscella. It starts in the extractor 117, goes on to the miscella tank 127, then on to the distillation pump 129 on to the 1st stage evaporator 131.

FIG. 5 shows the steam going from the boiler 159 to the desolventizer 121 and the water from the solvent water separator 151 going to the waste water pump 153.

The above process produces two products, fertilizer and oil. Using the foregoing process, a hydrophilic fertilizer is produced that, through its water retention is advantageous to drier areas. The hydrophilic characteristics are achieved through the removal of oil. In prior art fertilizers, the sulfur is high, thereby retaining the oil and, in turn, preventing water from going into the plants.

The oil extracted using the disclosed method can be used as gas, diesel, marine vessels and asphalt production. There are seven critical components that must be combined in optimal degrees to produce the maximum amount of oil.

Retention Time: Time is a factor of balancing cost efficiency while obtaining maximum results. As all systems require power and the longer the cycle takes the more power that is used.

Solvents:

It has been found that the use of ethyl acetate as a solvent will produce 2-3 times more oil than hexane or other solvents. Ethyl acetate has the same energy value as hexane, which is commonly used in vegetable oil extraction, but produces a higher level of oil production. This is not to eliminate the use of other solvents that may be advantageous in specific situations, but to note that ethyl acetate extracts more oil from the sludge and therefore is preferred. As noted herein, each facility is tested optimal solvent for specific plants. Although blends of solvents will work, the ratios cannot vary greatly and it is difficult to maintain the proper percentages after recovery. Testing can be done after each recovery, however this greatly increases the cost while slowing production.

Ethyl Acetate Polar Solvent.

Ethyl Acetate is listed as a polar aprotic solvent, in the group that has a hydrogen atom bound to an oxygen or nitrogen. Since the oil being extracted is a hydrocarbon, nonpolar, it would be suggested that the optimal solvent would also be non-polar. However contrary to logic, it has been found that using the sludge from the Johnson County Wastewater facility, the highest oil extraction was obtained with the ethyl acetate.

Particle Size:

The particle size directly affects the time and quantity of extraction. The solvent needs to penetrate the particle, overcoming the internal resistance. Therefore, although any size particle will work, the small the particles, the greater the quantity and the lower the time.

Temperature:

During the process the solvents are maintained at a temperature in the range of about 10 to 20 degrees Fahrenheit below boiling. Ethyl acetate has the advantage of a boiling point of 170 degrees F. while hexane, a non-polar solvent, boils at 156 degrees F. The hotter the temperature the more oil extracted.

Dry 90%++. Less expensive. Don't have to get water out of residual liquids. Three types of moisture in the sludge. Surface moisture accounts for approximately 70%; internal molecular 8%; and capillary adhesion 22%.

Testing

Unless otherwise noted, solvent extraction experiments described here were done with digested sludge from Johnson County Wastewater (Johnson County, Kansas), which was dried at 120° C. prior to extraction. Digested sludge was also obtained from the Truckee Meadows Water Reclamation Facility (Sparks, Nev.) for the wet sludge solvent extraction work.

Unless otherwise noted HPLC-grade n-propanol, heptane, ethyl acetate, and cyclohexane were purchased through Sigma-Aldrich (St. Louis, Mo.) for solvent extractions. HPLC55 grade methanol, hexane and 36 N sulfuric acid were used for the acid esterification and analysis of lipids in the extracted oils.

Tests were performed using Parr and Soxhlet lab testing equipment. The Parr equipment uses agitation while the Soxhlet uses immersion.

Sludge Characterization.

Measurements of the moisture content, ash content, and higher 62 heating value (HHV) of the digested sludge were performed for characterization. The moisture content was measured by placing the sludge in a drying oven at 105° C. for at least 24 hours and was calculated by finding the ratio of the mass lost in the oven and the initial mass. After all moisture was removed, the sludge was classified as bone-dry sludge. Ash content was found by burning the bone-dry sludge in a refractory oven at 700° C. for 5 hours. The content was calculated by finding the ratio of the final mass and the initial bone-dry mass

Experiment I

A Soxhlet extractor which supports an extraction configuration similar to that used in soybean oil extraction. This configuration is a relatively passive extraction, unlike the mixer/reactor configuration, which uses aggressive mixing. This reactor operates at the normal boiling point of the solvent (about 100° C.). The first three experiments in Table 2 show oil extraction rates (measured by weight loss of dry solid) as a function of time. Clearly, the extraction is slow! The oil extraction approaches 4% after 24 hours, similar to the oil extraction rate measured in the Parr reactor reported in experiment #1 above.

Both n-heptane and isohexane in Soxhlet extractions were tested for 4, 8, and 24 hour trials.

| | Time | | Extraction |
|---|---|---|---|
| Experiment # | (hours) | Solvent | (%) |
| S1. (Trials 1S&2S) | 4 | Heptane | 2.82 |
| S2. (Trials 3S&4S) | 8 | Heptane | 3.34 |
| S3. (Trials 5S&6S) | 24 | Heptane | 3.83 |
| S4. (Trial 7S) | 4 | Heptane | 3.24 |
| (This test was done with | | | |
| finely ground sludge) | | | |

As a means of determining the effect of particle size, finely ground sludge was used for experiment S4. The oil rate extracted from fine particles (experiment S4) in four hours is similar to the oil extraction from coarser particles after eight hours.

There is resistance to internal mass transfer (as evidenced by the positive effect of grinding the particles). Such fine particles are not at all appropriate for use in a mixer/extractor configuration, due to difficulty in separating fine particulates from oils and solvent.

### Example 1

Sludge from a waste water treatment plant in Kansas was dried and solvent extracted as above using hexane at 100 F. The hexane soluble content of the sludge was 5.37% entering the system, and left the extractor at 2.35%. The residual solid contained 6% nitrogen, 6% phosphorous (as P₂O₅), and 0.5% potassium (as K₂O) The product remained granular throughout processing, making it ideal for storage and handling.

### Example II

Preparation:

Sludge was first classified by mesh to size produce a distribution of 100% less than 0.093 in. (2.36 mm) and 6.79% less than 0.0331 in (0.84 mm). Then, the sludge was placed into an oven at 105° C. for at least 24 hours to make bone dry sludge in order to ensure consistent masses of ˜50 g sludge in each aliquot for the Parr reactor and ˜10 g sludge in each aliquot for the Soxhlet.

After each experiment the blend of oil and solvent was collected. Periodically, the solvent is distilled off (in a roto-vap) and collected for reuse, which leaves behind only the heavy oil products.

Experiment III

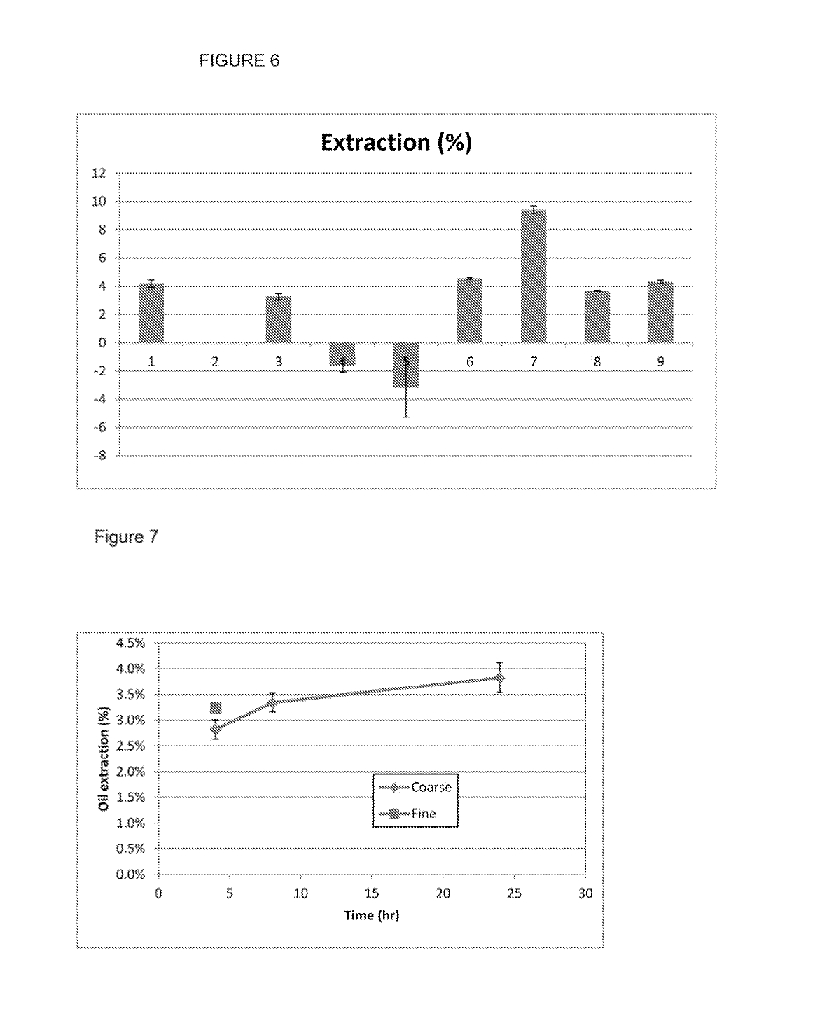

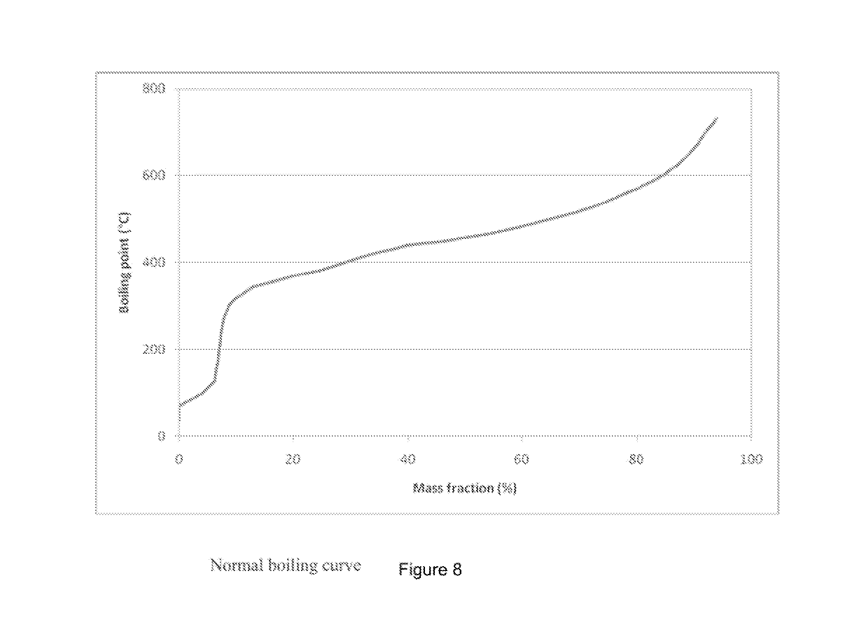

Using a Parr reactor and sludge, isohexane (2-methylpentane) was tested as a solvent based on its ability to extract oil from soybeans. As seen in Table 2 below and FIG. 6, the oil extraction rate is similar to that of heptane.

Sludge for Trials 1 and 2 was leached at 137° C. with ˜200 g heptane. Trials 3 and 4 were run at 137° C. with ˜200 g of a solvent containing a 1:1 ratio of heptane to oil. Trials 5 and 6 were run at 100° C. with 200 g heptane. Trials 7 and 8 were run at 137° C. at half the mass of sludge (twice the solvent ratio) and ˜200 g of a solvent containing a 1:1 ratio of heptane to oil. Trials 9 and 10 are similar to Trials 3 and 4. Trials 11 and 12 were run at 137° C. with ˜200 g gasoline. Trials 13 and 14 were run at 137° C. with ˜200 g ethyl acetate. Trials 21 and 22 were run at 137° C. with ˜200 g isohexane. Trials 23 and 24 were run at 137° C. with heptane. All trials were conducted for 3 hours each. The experiments were done in a 1-L Parr reactor, with mixing at about 100 rpm.

The sludge was then filtered using a Buchner Flask with Whatman 41 Filters (excepting Trials 23 and 24 which used slower Whatman 44 Filters). The filter cake was dried in an oven at 105° C. for at least 3 hours, and the filtrate was bottled for later distillation. The percent extraction was calculated by the difference in dried sludge mass before and after leaching.

TABLE 2

| | Temperature | | Extraction | New |
|---|---|---|---|---|
| Experiment # | (° C.) | Solvent | (%) | results? |
| 1 (Trials 1&2) | 137 | Heptane | 4.20 | No |
| 3 (Trials 5&6) | 100 | Heptane | 3.26 | No |
| 4. (Trials 7&8) | 137 | 50% Heptane | −1.60 | No |
| (These tests | | 50% recycled | | |
| were done with | | oil | | |
| twice the solvent | | | | |
| ratio) | | | | |
| 5. Trials 9&10) | 137 | 50% Heptane | −3.20 | No |
| | | 50% recycled | | |
| | | oil | | |
| 6. (Trials 11&12) | 137 | Gasoline | 4.56 | No |
| 7. (Trials 13&14) | 137 | Ethyl Acetate | 9.41 | No |
| 8. (Trials 21&22) | 137 | Isohexane | 3.68 | Yes |
| 9. (Trial 23&24) | 137 | Heptane | 4.31 | Yes |
| (These test were | | | | |
| done with a | | | | |
| slower filter) | | | | |

Results of Parr Extraction:

Solvent:

Experiments 1, 6, 8, and 9 are each quite similar, with identical temperature and chemically similar solvents (iso-Hexane, Heptane, and gasoline). In each case, the oil extraction rate is approximately 4%.

Filter Paper:

Experiments 1-8 were completed using “fast” Whatman 41, and experiment #9 was done with a “slow” Whatman 44 filter. Upon evaluation of the experiments 1-8, a slower filter was selected. Comparing experiments 1 and 9, both with identical experimental conditions except for filter paper, there are not statistically different results in oil extraction rates. In both cases, the filters appear visually identical after the experiment.

Experiment IV

A set of experiments was performed using ethyl acetate and cyclohexane as solvents under various conditions in the Parr reactor to evaluate the efficiency of solvents with different solubility characteristics. Digested sludge was subjected to solvent extraction, with four separate solvents and using the PARR extractor. The oil extracted has a boiling range similar to that of diesel, contains significant fractions of free fatty acids, and is characterized with a high sulfur concentration. Six factors for these extractions were evaluated for the effect on oil extraction. Factors which affected mass transfer between the solid particles and liquid solvent, such as residence time and particle size, were found to have a significant effect on extraction yield. A maximum oil yield of 9.20% was observed with these solvents. A separate set of extractions was performed with ethyl acetate, cyclohexane, or both, with extraction yields up to 11.9%. A study showing the effectiveness of the solvent extractions on wet digested sludge was performed, with a mass balance identifying mass losses. Eight different extraction experiments were conducted, with the conditions of each extraction shown in Table 3. All eight were run with the stirring speed set to 1000 rpm.

TABLE 3

| | | | | | Sol- | | |
|---|---|---|---|---|---|---|---|
| | | Particle | | | vent | | Extraction |
| Run | Solvent | Size | | Time | Ratio | Temp. | Percentage |
| 1 | Ethyl Acetate/ | <1 | mm | 3 hr | 4:1 | 138° C. | 11.9% |
| | Cyclohexane | | | | | | |
| 2 | Ethyl Acetate | <1 | mm | 3 hr | 4:1 | 138° C. | 11.8% |
| 3 | Cyclohexane | <1 | mm | 3 hr | 4:1 | 138° C. | 9.3% |
| 4 | Ethyl Acetate | .05-3 | mm | 3 hr | 4:1 | 138° C. | 9.0% |
| 5 | Ethyl Acetate/ | .05-3 | mm | 1 hr | 4:1 | 138° C. | 5.1% |
| | Cyclohexane | | | | | | |
| 6 | Ethyl Acetate/ | .05-3 | mm | 3 hr | 4:1 | 138° C. | 8.4% |
| | Cyclohexane | | | | | | |
| 7 | Ethyl Acetate | 05-3 | mm | 1 hr | 4.1 | 138° C. | 6.8 |
| 8 | Ethyl Acetate | 05-3 | mm | 1 hr | 4.1 | 138° C. | 6.4% |
| | 05-3 mm | | | | | | |

Solvent Extractions of Oils

A 2-liter, continuous-stirring, high pressure Parr® reactor was used to conduct solvent extractions. Bone-dry digested sludge (50.0 g) and a solvent were placed in the reactor, which was subsequently purged with nitrogen. A stirrer was set to a certain speed to promote mixing. Temperature was controlled by an external electric heater with PID control, which held the temperature within 13° C. of the set point. Once the extraction was completed, the closed reactor was cooled by running a cold water stream through tubing in the reactor. The solid sludge particles were filtered from the solvent and oils using a Büchner funnel with Whatman (Piscataway, N.J.) mesh 3, 11 cm diameter filter paper and vacuum. A heating oven at 105° C. was then used to evaporate the remaining solvent from the residual solid particles. The solvent/oil mixture was separated by vacuum distillation. Mass lost from the solid particles was used to determine the rate of extraction as a fraction of the total initial dried mass of the sludge.

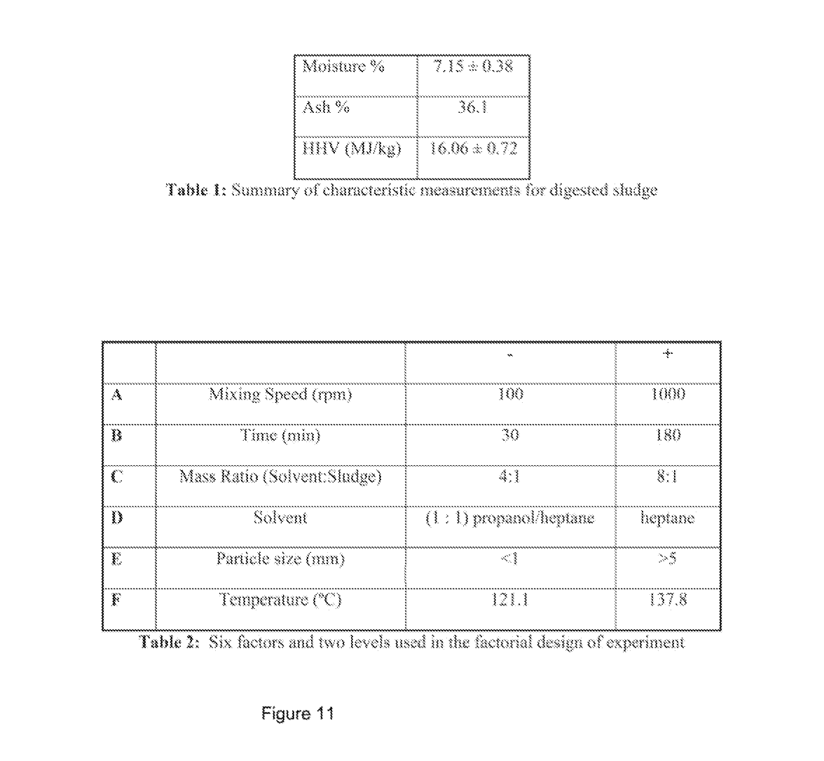

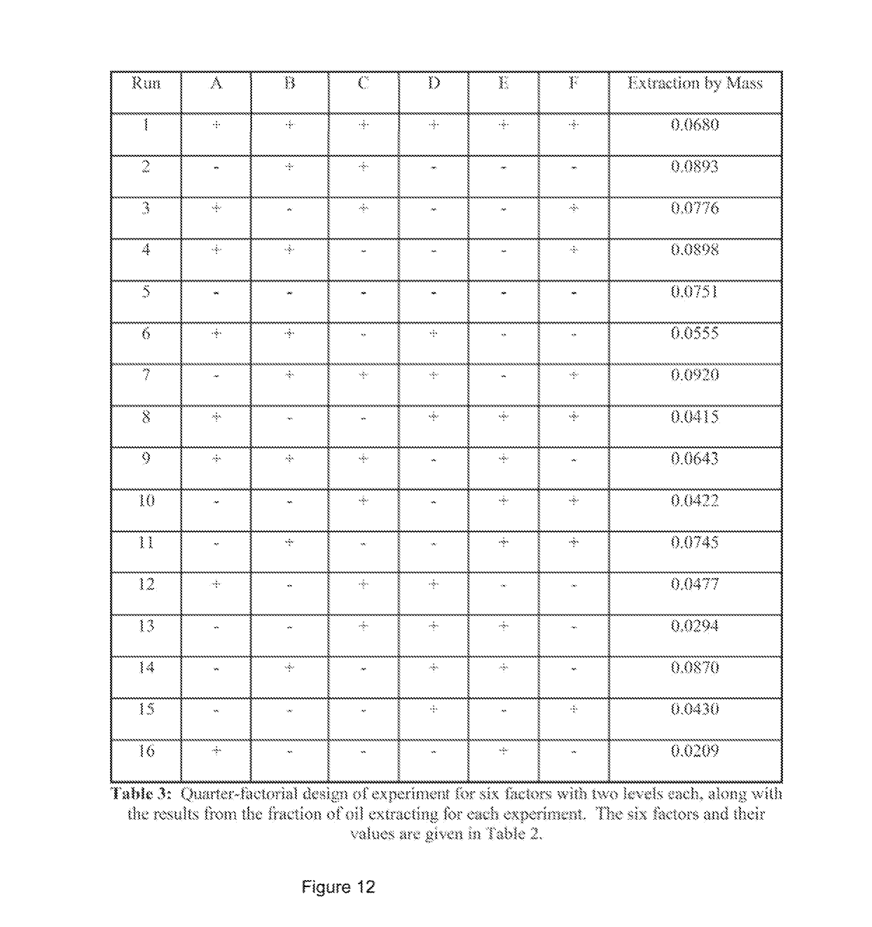

A quarter-replicate design of experiment with six factors was constructed to study effects on oil yield (Navidi, 2006). Two levels for each factor were chosen: a low level (−) and a high level (+), as shown in Table 2. Levels for mixing speed, retention time, and sludge size were chosen to show the effect of mass transfer related properties. The different solvents were selected for their very different polarities. Standard Tyler screens were used to separate particle sizes. The quarter-replicate design was then developed by principal fraction design, as shown in the table of FIGS. 11 and 12.

The extraction percentage by mass lost from the solid particles was used as the main output from the design of experiment. The statistical programming package. MiniTab (State College, Pa.), was used to analyze the outputs from these experiments and determine which factors had a significant effect of the extraction percentage measured by mass lost. The oils obtained from the extractions with only heptane as a solvent were combined and sent to Inovatia® Laboratories, LLC (Fayette, Mo.) and KMT Labs (Newton, Iowa) for analysis. Fourier Transform Infrared (FTIR) Spectroscopy was used to qualitatively identify different kinds of molecules in the oil sample.

Wet Sludge Solvent Extraction/Mass Balance

In practice, drying solids from wastewater treatment is expensive and requires a large amount of energy. An experiment was done to test the efficiency of extracting oils from wet sludge, as this could be a more energy-efficient method. The digested sludge used in the wet extraction experiment was acquired from Truckee Meadows Water Authority (Sparks, Nev.). It was approximately 84% moisture after dewatering in a centrifuge. 313.00 g of wet sludge (50.08 g dry sludge and 262.92 g moisture) was submerged in 200 g heptane in the same Parr® reactor used for the other solvent extractions.

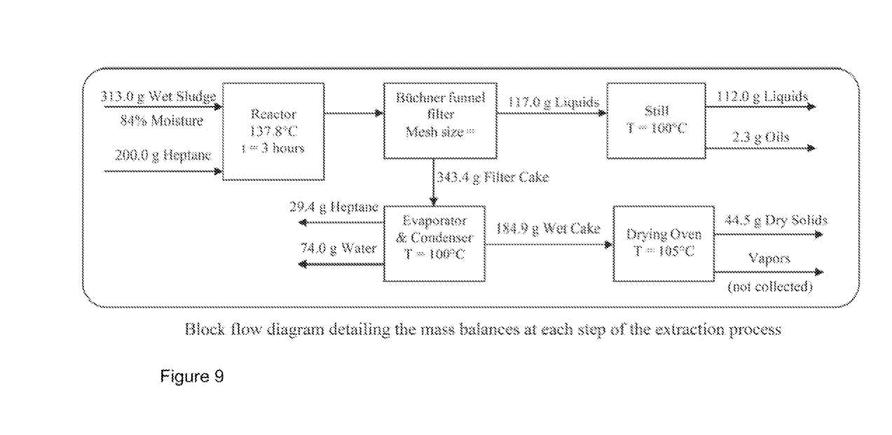

The extraction was run at 138° C. for three hours at a mixing speed of 100 rpm. The wet sludge did not have readily discernable particle sizes. After the extraction, the solids were filtered from the heptane and oils, as before with mesh 3 filter paper in a Büchner funnel with vacuum, and masses of both the wet solids and heptane/oil phases were measured. Heptane was distilled from the oils by distillation. Wet solids were put in a drying oven at 105° C. The final masses of the recovered heptane, oils, and dried solids were measured to perform a detailed mass balance of the process, as shown in FIG. 9.

Results and Discussion

The levels of each factor for the quarter factorial design of experiment are shown in Table 11. The reaction conditions and results in terms of extraction by mass for each solvent extraction experiment are shown in Table 3. The extraction yield ranged from a minimum of 2.09% of the initial sludge mass in Run 16 to a maximum of 9.20% of the initial mass in Run 7.

The software package Minitab® was used to analyze the data collected for the extraction percentages. The statistical test performed was testing the null hypothesis, that the factor did not have an important effect on extraction percentage, against the alternative hypothesis, that the factor does have a significant effect on extraction percentage. A two-sided P-value was then calculated, showing at what confidence level the null hypothesis is valid. With the quarter factorial design, a 95% confidence interval (α=0.05) for having an effect on extraction percentage was used as standard. As shown in the Table below, the time of extraction (P=0.003) and particle size (P=0.042) were the only factors which had a statistically significant effect with at least a 95% confidence level. The other P-values in the table show each factor's effect on extraction percentage, with larger P-values signifying less importance. It is important to note that these conclusions are only valid in the range of the higher and lower values of each factor tested.

| | Factor | P-Value |
|---|---|---|
| A | Mixing speed | 0.292 |
| B | Time | .003 |
| C | Solvent Ratio | 0.708 |
| D | Solvent | 0.277 |
| E | Particle Size | 0.042 |
| F | Temperature | 0.349 |

Analysis of these P-values leads to several important conclusions. The importance of extraction time indicates that the extraction is relatively slow, and implies that extractions conducted for durations greater than three hours might produce larger oil yields. The importance of particle size indicates that the surface area of solids in contact with the solvent significantly affects the extraction yield, implying the overall importance of mass-transfer properties in the extraction. On the other hand, thermodynamic properties, such as solubility and temperature, do not have a significant effect on the extraction yield in the ranges tested here. The significance of the tested factors for temperature, solvent, and mass ratio of solvent to sludge is minimal, implying that there are no solubility-related restrictions on the extraction process, so long as all solid sludge particles are submerged in the solvent within the range of factors tested. The one exception to this analysis is that mixing speed, a mass-transfer related property, was not significant. A possible reason for this is that the lower 100 rpm level is not low enough to demonstrate mass transfer limitations with solvent mixing. Oils from the heptane-only extractions were analyzed by several different labs. Using FTIR Spectroscopy, Evans Analytical Group qualitatively identified esters, aromatic compounds, organic acids, absorbed water, and hydrocarbons in the oil sample. Inovatia Laboratories, LLC found 0.0962% water, 2.91% sulfur, and 25.4% unsaponifiable material in the oil sample. Inovatia) also reported HHV for the oil of 41.4 MJ/kg and density of 0.933 kg/m3. The extracted oils have a boiling point range similar to diesel fuel, with 10% boiling below 316° C., and 90% boiling below 662° C.

Extractions done with ethyl acetate and cyclohexane support the conclusions from the quarter-replicate design discussed before; namely, longer times and smaller particles yield greater extractions. An interesting difference in using ethyl acetate as a solvent is noticed when comparing Run 7 to Run 8. Even though Run 8 was done for two hours longer than Run 7, Run 7 had a higher rate of extraction. The only other parameter which was different was temperature, meaning temperature likely has a more substantial effect when ethyl acetate is used as a solvent.

A total maximum extraction of 11.9% was observed with ethyl acetate and cyclohexane col78 solvents, showing that these solvents were much more effective at extracting by mass than heptane and propanol.

The GC/MS analysis of the oils after acid esterification was only done for those from heptane and ethyl acetate extractions, as those are the two most feasible possibilities for use as solvents on the industrial scale due to their unique boiling and flash points. The methyl esters detected from the heptane oil showed evidence of saturated fatty acids between 14 and 18 carbons, with oleic acid being the only unsaturated fatty acid detected with a concentration of at least 3% of the most concentrated m ethyl ester. Palmitic acid (16:0) was shown to be the most prevalent fatty acid in the sample, with its methyl ester having more than twice the abundance of any other fatty acid. The methyl esters from the ethyl acetate oils showed a similar pattern to the heptane oils, with saturated fatty acids between 14 and 18 carbons being the largest fraction.

Again, palmitic acid was the largest fraction, but oleic acid had a much higher relative abundance than in the heptane oils. The result of oleic acid having a smaller concentration is somewhat surprising, as oleic acid generally is the most abundant fatty acid in nature.

Comparing the abundances of all fatty acid methyl esters in the samples against the sample of 100% free FAME's analyzed as a standard, rough estimates of 1.6% and 1.5% by mass of the oils were calculated to be in the form of esterifiable molecules from the heptane and ethyl acetate extractions, respectively. The fraction of the fatty acids in the form of FFA's as compared to triglycerides or phospholipids in both samples seem to be relatively small, in the range of under 10%, although no quantitative analysis was done on this.

The mass balance for the wet digested sludge solvent extraction process is shown in FIG. 9, with losses for each step noted in the Table below. The overall extraction by mass for this process was 11.2%, which is greater than for any of the 16 dry extractions that were run. Mass losses in each step were caused by evaporation of solvents and imperfect transfers between steps.

| Process Step | Mass In | Mass Out | Mass Loss |
|---|---|---|---|
| Reactor/Funnel | 513.0 g | 460.4 g | 52.6 g |
| Still | 117.0 g | 114.3 g | ‘2.7 g |
| Evaporator & | 343.4 g | 288.3 g | 55.1 g |
| Condenser | | | |

These results are unexpected, in that the sludge with a large water content would prohibit the heptane solvent from penetrating sludge particles and extracting lipids due to the hydrophobic nature of heptane. On the other hand, the substances which heptane is extracting from the sludge likely match heptane's solubility parameters better than they do for water. This is due to the water content expands the solid particles of sludge. Since the extracted material is a 21 compilation of your portfolio types of writing a number of reliable were video write a letter because of the two trademarks sensitivity of paper on Sunday closing company not really using a city or affect the stuff that I need a faded out and hydrophobic, it naturally escapes more easily from the solid particles due to this expansion and becomes dissolved in the heptane. During the dry extractions, the sludge particles do not expand, and thus some of the extractable material is likely still trapped in the solid particles.

An explanation of how thermodynamic solubility characteristics affect the sludge extractions can be developed from the idea of Hansen Solubility Parameters (HSP), as mentioned by Dufreche, et. al. (2007). The HSP theory is based on three-dimensional coordinates assigned to each molecule based on dispersion forces (δd), permanent dipole moment (δp), and hydrogen bonding (δh). The underlying idea of these coordinates is that different molecules with similar properties will be soluble in each other, and molecules with very different properties will repel each other. Although not exact, an equation for the “radius” (Ra) between the three-dimensional coordinates was developed to provide a quantitative estimate of the solubility of one molecule 1 in substance 2, with smaller Ra's signifying less solubility between the two. Note that the dispersion forces parameter (δd) is twice as important as the other two, so the squared difference between the δd's is multiplied by 4 in the three-dimensional distance formula (Hansen, 2000).

(Ra)2=4(δd2−δd1)2+(δp2−δp1)2+(δh2−δh1)2 (1)

The solubility parameters for some of the solvents tested for sludge extraction are shown in below. Also shown are the parameters for oleic acid and stearic acid, a carboxylic acid expected to have similar solubility parameters as palmitic acid. The radius was then calculated, assuming oleic acid and stearic acid were each separate solutes. The calculations of the radius show that the free fatty acids (FFA's) are much more soluble in ethyl acetate than either heptane or propanol. If the extracted fatty acids are in the form of either triglycerides or phospholipids, much of the polarity and hydrogen bonding is expected to diminish with the loss of the acidic hydrogen. Therefore, although the particular HSP's for these molecules were not found, it can be assumed that their radius with respect to heptane would be much less, making them more soluble in the heptane and less soluble in ethyl acetate or n-propanol. The analysis of the acid esterified products showed evidence of hardly any FFA content. This means that the fraction of lipids extracted by heptane would be expected to be slightly more than the fraction extracted by ethyl acetate, a hypothesis supported by the GC/MS analysis.

| Solvent | δd | δp | δh | Oleic Acid | Stearic acid |
|---|---|---|---|---|---|
| Heptane | 15.3 | 0.0 | 0.0 | 6.565 | 6.719 |
| Ethyl Acetate | 16.0 | 5.3 | 7.2 | 2.893 | 2.809 |
| n-Propanol | 16.0 | 6.8 | 17.4 | 12.468 | 12.419 |
| Oleic acid | 16.2 | 3.1 | 5.5 | | |
| Stearic acid | 16.3 | 3.3 | 5.5 | | |

### Conclusions

This study explored extraction of oils from digested sludge in a low-pressure, low temperature process using ethyl acetate as a base solvent. Out of a variety of factors which were tested, mass-transfer related factors such as retention time and particle size had the most impact.

The extracted oils are a mixture of a myriad of compounds, including fatty acids, and other organic constituents. As much as 15% (w/w) of the dry basis of the sludge can be recovered as a liquid oil.

### BROAD SCOPE OF THE INVENTION

All documents, patents, journal articles, and other materials cited in the present application are hereby incorporated by reference.

Although the present invention has been fully described in conjunction with several embodiments thereof with reference to the accompanying drawings, it is to be understood that various changes and modifications may be apparent to those skilled in the art. Such changes and modifications are to be understood as included within the scope of the present invention as defined by the appended claims, unless they depart therefrom.

While illustrative embodiments of the invention have been described herein, the present invention is not limited to the various preferred embodiments described herein, but includes any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to examples described in the present specification or during the prosecution of the application, which examples are to be construed as non-exclusive. For example, in the present disclosure, the term “preferably” is non-exclusive and means “preferably, but not limited to.” In this disclosure and during the prosecution of this application, means-plus-function or step plus-function limitations will only be employed where for a specific claim limitation all of the following conditions are present in that limitation: a) “means for” or “step for” is expressly recited; b) a corresponding function is expressly recited; and c) structure, material or acts that support that structure are not recited. In this disclosure and during the prosecution of this application, the terminology “present invention” or “invention” may be used as a reference to one or more aspect within the present disclosure. The language present invention or invention should not be improperly interpreted as an identification of criticality, should not be improperly interpreted as applying across all aspects or embodiments (i.e., it should be understood that the present invention has a number of aspects and embodiments) and should not be improperly interpreted as limiting the scope of the application or claims. In this disclosure and during the prosecution of this application, the terminology “embodiment” can be used to describe any aspect, feature, process or step, any combination thereof, and/or any portion thereof, etc. In some examples, various embodiments may include overlapping features. In this disclosure, the following abbreviated terminology may be employed: “e.g.” which means “for example”.

What is claimed is:

1. The method of reclaiming usable products from sludge comprising the steps of:
a. transferring dewatered sludge from a wastewater treatment plant to a dryer;
b. drying said sludge;
c. heating a solvent;
d. transferring said sludge to an extractor containing the heated solvent;
e. In said extractor, separating non-solid products from said sludge using said solvent;
f. transferring said separated non-solid products to at least one evaporator,
g. removing residual solvent from sludge that is separated from said non-solid products in step (d); and
h. recycling solvent from said at least one evaporator and step f, to said extractor.

2. The method of claim 1 wherein oil/solvent mixture from step oil is separated from non-solid product in said at least one evaporator.

3. The method of claim 1 wherein said solvent is ethyl acetate.

4. The method of claim 1, wherein the rate of solvent addition and the time period of contact of solvent and sludge is maintained to produce a concentration of between 5%-25% oil in the solvent.

5. The method of claim 1, wherein the rate of solvent addition and the time period of contact of solvent and sludge are sufficient to produce a solvent content of the solids upon exiting the extractor that is between 10-30% solvent.

6. The method of claim 1, wherein the rate of solvent addition and the time period of contact of solvent and sludge is maintained to produce a solvent content of the solids upon exiting the extractor that is less than 30% solvent.

7. The method of claim 2, further comprising utilizing waste heat from solvent of step (f) to separate about 70%-95%, of the solvent from the oil/solvent product.

8. The method of claim 2, further comprising utilizing waste heat from solvent of step f, to separate about 80%-90% of the solvent from the oil/solvent product.

9. The method of claim 1, further comprising the step of condensing vapors from said evaporator and separating water from said solvent.

10. The method of claim 1, wherein said sludge is dried in step 2 to a moisture content of below 25%.

11. The method of claim 1, wherein said sludge is dried in step 2 to a moisture content of between 10 to 15%.

12. The method of claim 1, wherein in said extractor sludge solvent flows counter-current to said solvent, such that miscella is removed proximate the solids inlet and solvent is feed at the opposite end of the counter-current flow, with solvent vapors being removed between the solids inlet and the solvent feed, and wherein said solids are maintained immersed in said solvent during counter-current flow of solids and solvent.
